(19) United States
(12) Patent Application Publication
(10) Pub. No.: US 2011/0039898 A1
(43) Pub. Date: Feb. 17, 2011

(54) METHOD FOR SCAVENGING FREE RADICALS AND INHIBITING TYROSINASE AND MELANIN

(76) Inventors: Chao-Hsiang CHEN, Taoyuan County (TW); Mei-Yin Chien, Tao-Yuan County (TW); Wen-Chi Hou, Taipei City (TW); Yin-Hsiu Lin, Taipei County (TW)

Correspondence Address: Muncy, Geissler, Olds & Lowe, PLLC, 4000 Legato Road, Suite 310, FAIRFAX, VA, 22033, US

(21) Appl. No.: 12/609,718

(22) Filed: Oct. 30, 2009

(30) Foreign Application Priority Data
Aug. 17, 2009 (TW) 098127575

(51) Int. Cl. A61K 31/4406; A61K 31/44; A61P 43/00

(52) U.S. Cl. 514/355

(57) ABSTRACT

A method for scavenging free radicals and inhibiting tyrosinase and melanin is provided. The method includes administering to a subject of effective amounts in a pharmaceutical composition comprising a compound of Formula (I) or (II) and a pharmaceutical acceptable salt, metal salt or solvent.

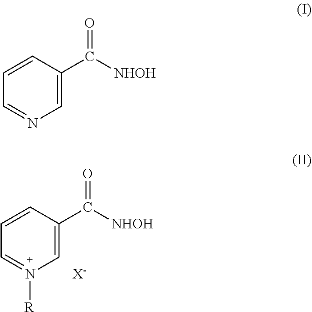

In Formula (II), R is C1-6 alkyl, vitamin C or sugar. X is chlorine, bromine or iodine. The subject is a human. The pharmaceutical composition is for external and internal use.

### CROSS REFERENCE TO RELATED APPLICATIONS

This Application claims priority of Taiwan Patent Application No. 098127575, filed on Aug. 17, 2009, the entirety of which is incorporated by reference herein.

### BACKGROUND OF THE INVENTION

1. Field of the Invention

The invention relates to a method, and more particularly to a method for scavenging free radicals and inhibiting tyrosinase and melanin.

2. Description of the Related Art

Currently, anti-free radical products comprise coenzyme Q10, vitamin C, vitamin E, vitamin B complex, flavonoid, vitamin A and β-carotene, with strong reducibility to effectively scavenge free radicals. The anti-free radical products can be prepared, such as healthy foods, in external and/or internal forms to prevent free radical damage, slowing the aging process. However, antioxidants, such as vitamin C or vitamin E, are unstable.

According to a melanin formation mechanism, tyrosinase is a key enzyme in the rate determining step of melanin formation. Thus, tyrosinase inhibitors are utilized in skin care. Skin care products comprise tretinoin or retinoic acid from unripe apple and lemon (Briganti et al., 2003; Solano et al., 2006), Arbutin separated from bearberry leaf (Hori et al., 2004), kojic acid separated from fermentation process of malting rice (Kasraee et al., 2004), ellagic acid from strawberry and apple (Shimogaki et al., 2000; Briganti et al., 2003) and C2-ceramide from animals (Kim et al., 2001; Kim et al., 2002; Kim et al., 2005). However, kojic acid have been reported to be a carcinogenic substance (Fujimoto et al., 1998). Thus, development of safe skin care products, from sources without carcinogenic properties and toxicities is desirable.

### BRIEF SUMMARY OF THE INVENTION

One embodiment of the invention provides a method for scavenging free radicals, and inhibiting tyrosinase and melanin comprising administering to a subject of effective amounts in a pharmaceutical composition comprising a compound of Formula (I) or (II) and a pharmaceutical acceptable salt, metal salt or solvent.

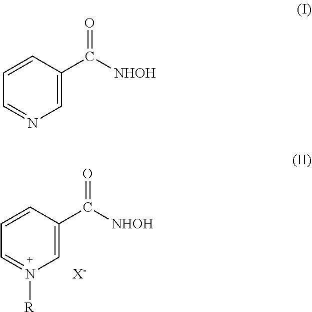

In Formula (II), R is C1-6 alkyl, vitamin C or sugar. X is chlorine, bromine or iodine.

The compound of Formula (I) provided by the invention is nicotinyl hydroxamic acid.

The nicotinyl hydroxamic acid (N-hydroxy-3-pyridine carboxamide, nicotinohydroxamic acid, nicotinylhydroxamic acid, nicoxamat, and pyridine-3-carbohydroxamic acid or 3-pyridine hydroxamic acid), is capable of antioxidation and inhibition to tyrosinase, and can serve as a melanin inhibitor to be applied in cosmetics and medicine. Additionally, the nicotinyl hydroxamic acid also inhibits polyphenol oxidase, and can be applied in food preservation.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawing, wherein:

FIG. 1 shows a relationship between the nicotinyl hydroxamic acid concentration, the N-methyl nicotinic acid hydroxamate iodide concentration and DPPH free radical scavenging activity according to an embodiment of the invention.

FIG. 2 shows a relationship between the nicotinyl hydroxamic acid concentration and superoxide free radical scavenging activity according to an embodiment of the invention.

FIG. 3 shows a relationship between the nicotinyl hydroxamic acid concentration, the N-methyl nicotinic acid hydroxamate iodide concentration and tyrosinase inhibition (substrate is L-tyrosine) according to an embodiment of the invention.

FIG. 4 shows a relationship between the nicotinyl hydroxamic acid concentration, the N-methyl nicotinic acid hydroxamate iodide concentration and tyrosinase inhibition (substrate is L-DOPA) according to an embodiment of the invention.

FIG. 5 shows a relationship between the nicotinyl hydroxamic acid concentration, the N-methyl nicotinic acid hydroxamate iodide concentration and tyrosinase activity (without addition of MSH) according to an embodiment of the invention.

FIG. 6 shows a relationship between the nicotinyl hydroxamic acid concentration, the N-methyl nicotinic acid hydroxamate iodide concentration and tyrosinase activity (addition of MSH) according to an embodiment of the invention.

FIG. 7 shows a relationship between the nicotinyl hydroxamic acid concentration, the N-methyl nicotinic acid hydroxamate iodide concentration and melanin content (without addition of MSH) according to an embodiment of the invention.

FIG. 8 shows a relationship between the nicotinyl hydroxamic acid concentration, the N-methyl nicotinic acid hydroxamate iodide concentration and melanin content (addition of MSH) according to an embodiment of the invention.

FIG. 9 shows a relationship between the nicotinyl hydroxamic acid concentration, kojic acid concentration and melanin content (without addition of MSH) according to an embodiment of the invention.

FIG. 10 shows a relationship between the nicotinyl hydroxamic acid concentration, kojic acid concentration and melanin content (addition of MSH) according to an embodiment of the invention.

FIG. 11 shows a relationship between the nicotinyl hydroxamic acid concentration and cell viability of human melanocytes and melanoma cells (B16-F10) according to an embodiment of the invention.

FIG. 12 shows a relationship between the N-methyl nicotinic acid hydroxamate iodide concentration and cell viability of human melanocytes and melanoma cells (B16-F10) according to an embodiment of the invention.

FIG. 13 shows the variation of the absorbance of the apple juice containing the nicotinyl hydroxamic acid with various concentrations within two hours according to an embodiment of the invention.

FIG. 14 shows the variation of the absorbance of the apple juice containing the N-methyl nicotinic acid hydroxamate iodide with various concentrations within two hours according to an embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

One embodiment of the invention provides a method for scavenging free radicals, and inhibiting tyrosinase and melanin comprising administering to a subject of effective amounts in a pharmaceutical composition comprising a compound of Formula (I) or (II) and a pharmaceutical acceptable salt, metal salt or solvent.

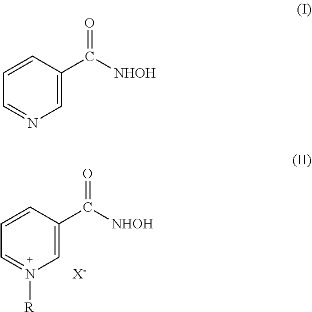

In Formula (II), R may be C1-6 alkyl, vitamin C or sugar. X may be chlorine, bromine or iodine.

In Formula (II), the sugar may comprise disaccharide such as sucrose, maltose or lactose, pentose such as D,L-xylose, D,L-rhamnose, D,L-ribose or D,L-arabinose, or hexose such as D,L-glucose, D,L-galactose, D,L-mannose, D,L-fructose, galacturonic acid, glucouronic acid, glucosamine or N-acetylglucosamine.

The subject may be a human. The pharmaceutical composition may be for external use or internal use.

The compound of Formula (I) provided by the invention is nicotinyl hydroxamic acid.

The mechanism for inhibition of tyrosinase by nicotinyl hydroxamic acid is described as follows. Tyrosinase is the most important enzyme during melanogenesis, regulating and catalyzing two initial rate-limiting steps, for example, tyrosine hydroxylation and dopa oxidation (Riley, 1993). Additionally, tyrosinase-related protein 1 (TRP-1, DHICA oxidase) oxidizes 5,6-dihydroxyindole-2-carboxylic acid (DHICA) to indole-5,6-quinone-2-carboxylic acid. Tyrosinase-related protein 2 (TRP-2, dopachrome tautomerase (DCT)) transfers dopachrome into DHICA (Riley, 1997; Busca and Ballotti, 2000; Ortonne and Ballotti, 2000; Wakamatsu et al., 2002). Melanogenesis is completed through a series of oxidation and polymerization steps.

After using nicotinyl hydroxamic acid to treat B16-F10 cells for 24 hours, proteins are extracted and analyzed using the western blot method. The contents of tyrosinase and TRP-1 are reduced as the concentration of nicotinyl hydroxamic acid is increased. In accordance with the analytical results, the content of TRP-2 is irrelevant to the concentration of nicotinyl hydroxamic acid.

Microphthalmia-associated transcription factor (MITF) is a transcription factor controlling tyrosinase and TRP-1 expression in cells, with a helix-loop-helix leucine zipper structure, capable of regulation of melanogenesis and survival and proliferation of melanocytes. According to the analytical results obtained using the western blot method, it was shown that the content of MITF is also reduced as the concentration of nicotinyl hydroxamic acid is increased.

The mechanism of inhibition of melanogenesis by nicotinyl hydroxamic acid in B16-F10 cells is further described as follows. ERK and AKT/PKA pathways participate in melanogenesis. PD98059, a MEK selective inhibitor, inhibits the activity of ERK (ERK is activated by MEK). LY294002, a phosphatidylinositol 3-kinase (PI3K) inhibitor, blocks the AKT/PKB pathway. The two inhibitors are used to determine the participation of nicotinyl hydroxamic acid in the ERK and AKT/PKA pathways. The analytical results indicate that adding PD98059 and LY294002 increases melanin content in cells. However, when nicotinyl hydroxamic acid is simultaneously added, the inhibition of melanin thereby is reduced by PD98059 and LY294002.

Additionally, according to the analytical results obtained using the western blot method, it was shown that the expression of p-ERK, p-MEK and p-AKT is increased as the concentration of nicotinyl hydroxamic acid is increased. Thus, in addition to inhibition of tyrosinase and adjustment of MITF, nicotinyl hydroxamic acid also reduces the melanin content in cells through ERK and AKT/PKB pathways.

The nicotinyl hydroxamic acid (N-hydroxy-3-pyridine carboxamide, nicotinohydroxamic acid, nicotinylhydroxamic acid, nicoxamat, and pyridine-3-carbohydroxamic acid or 3-pyridine hydroxamic acid), is capable of antioxidation and inhibition to tyrosinase, and can serve as a melanin inhibitor to be applied in cosmetics and medicine. Additionally, the nicotinyl hydroxamic acid also inhibits polyphenol oxidase, and can be applied in food preservation.

### EXAMPLES

The B16-F10 cell was purchased from the Food Industry Research and Development Institute (Taiwan). Human Epidermal Melanocytes and Human Epidermal Keratinocytes (neonatal) were purchased from Cascade Biologics. Nicotinyl hydroxamic acid, Kojic acid, Arbutin, MIT, Tyrosinase, DPPH, Pyrogallol, α-MSH, Tyrosine, DOPA, RIPA solution, 2-mercaptoethanol, PD98059 and LY294002 were purchased from Sigma. Medium 254, EpiLife, Human Melanocyte Growth Supplement and Human Keratinocyte Growth Supplement were purchased from Cascade Biologics. Dulbecco's Modified Eagle Medium (DMEM) was purchased from GIBCO. Fetal Bovine Serum (JRH bioscience) was purchased from Sigma. Dish and Flask were purchased from CORNING. Antibody: TRP1 (G-17) goat polyclonal IgG, Tyrosinase (M-19) goat polyclonal IgG, TRP2 (G-15) goat polyclonal IgG and AKT (C-20) goat polyclonal IgG were purchased from Santa Cruz biotechnology, Inc. Antibody: MITF rabbit polyclonal IgG was purchased from Abacm. Anti-body: phosphor-MEK1/2 (Ser 217/221) rabbit polyclonal IgG, p44/42 MAP kinase rabbit polyclonal IgG, phospho-p44/42 (Thr 202/Tyr 204) rabbit polyclonal IgG, MEK rabbit polyclonal and Phospho-Akt (Ser 473) rabbit polyclonal IgG were purchased from Cell Signaling Technology, Inc. Antibody: Goat Anti-rabbit IgG, HRP conjugate, Rabbit Anti-goat IgG and HRP conjugate were purchased from Sigma. BCA™ protein assay kit was purchased from Thermo scientific.

### Example 1

Free Radical Scavenging Assay

DPPH Free Radical Scavenging Assay

300 μl of a sample solution (nicotinyl hydroxamic acid and N-methyl nicotinic acid hydroxamate iodide) with various concentrations, 100 μl of a Tris-HCl buffer (1M, pH 7.9) and 600 μl of a DPPH/MeOH (100 μM) were sequentially added to a 1.5 ml centrifugal tube, uniformly mixed and reacted for 20 minutes under room temperature and a dark environment. The result was then measured by a spectrophotometer (517 nm).

Superoxide Free Radical Scavenging Assay

80 μl of a sample solution with various concentrations, 80 μl of substrate buffer containing 50 mM Tris-HCl buffer (pH 8.3) and 1 mM EDTA, and 40 μl of pyrogallol (1.5 mM, dissolved in 10 mM HCl) were sequentially added and uniformly mixed. The result was then measured by a spectrophotometer (420 nm).

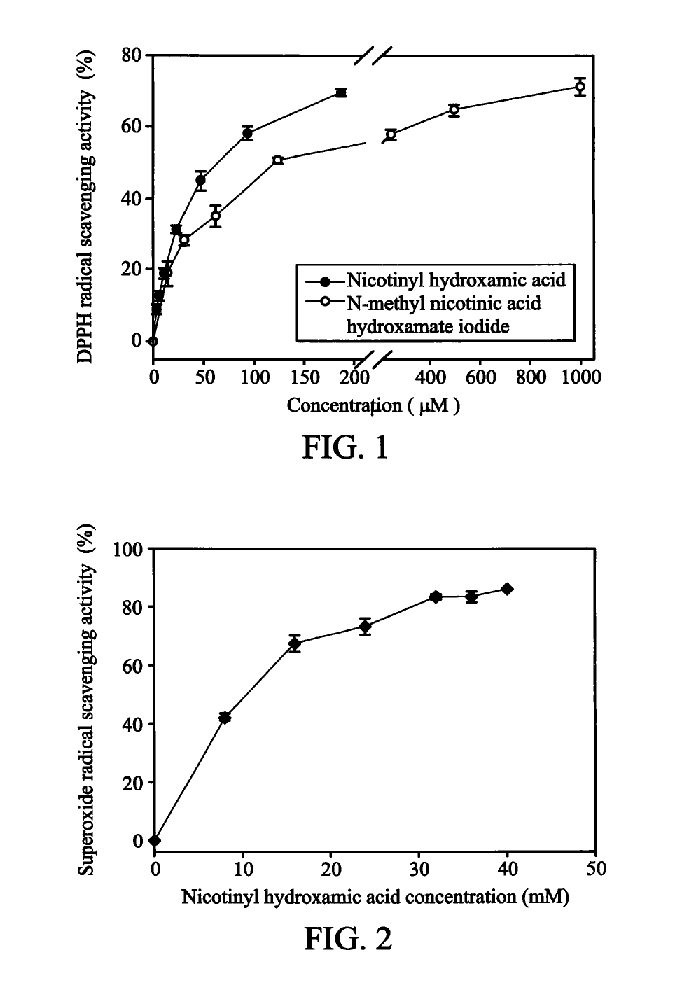

FIG. 1 shows a relationship between the nicotinyl hydroxamic acid concentration, the N-methyl nicotinic acid hydroxamate iodide concentration and DPPH free radical scavenging activity. FIG. 2 shows a relationship between the nicotinyl hydroxamic acid concentration and superoxide free radical scavenging activity. The results indicated that the IC₅₀ of the nicotinyl hydroxamic acid for scavenging DPPH free radicals was 65.81 μM and that for scavenging superoxide free radicals was 12.31 mM. The IC₅₀ of the N-methyl nicotinic acid hydroxamate iodide for scavenging DPPH free radicals was 125.2 μM.

### Example 2

Tyrosinase Inhibition Assay

40 μl of a sample solution (nicotinyl hydroxamic acid and N-methyl nicotinic acid hydroxamate iodide) with various concentrations, 100 μl of a phosphate buffer (250 mM, pH 6.5), 50 μl of a L-tyrosine (5 mM) or L-DOPA, and 5 μl of a tyrosinase (1000 unit/ml) were sequentially added to a 96-well plate, uniformly mixed and reacted in a 37° C. culture box. The absorption of the result was then measured by an ELISA reader (475 nm).

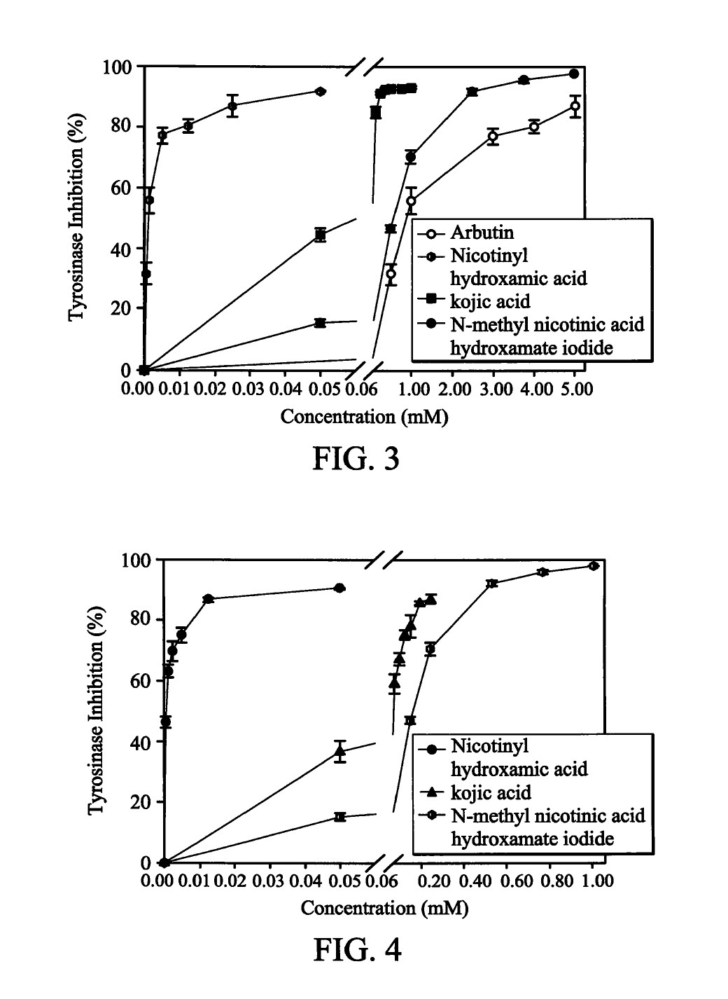

FIG. 3 shows a relationship between the nicotinyl hydroxamic acid concentration, the N-methyl nicotinic acid hydroxamate iodide concentration and tyrosinase inhibition (substrate is L-tyrosine). FIG. 4 shows a relationship between the nicotinyl hydroxamic acid concentration, the N-methyl nicotinic acid hydroxamate iodide concentration and tyrosinase inhibition (substrate is L-DOPA). The results indicated that both of the nicotinyl hydroxamic acid and N-methyl nicotinic acid hydroxamate iodide effectively inhibited tyrosinase activity for the L-tyrosine substrate and the L-DOPA substrate. When using L-tyrosine as the substrate, the IC₅₀ for Arbutin for inhibiting tyrosinase was 0.877 mM, the IC₅₀ for kojic acid for inhibiting tyrosinase was 0.05977 mM, the IC₅₀ for nicotinyl hydroxamic acid for inhibiting tyrosinase was 1.066×10⁻³ mM and the IC50 for N-methyl nicotinic acid hydroxamate iodide for inhibiting tyrosinase was 0.5642 mM. When using the L-DOPA as the substrate, the IC₅₀ for kojic acid for inhibiting tyrosinase was 0.0945 mM, the IC₅₀ for nicotinyl hydroxamic acid for inhibiting tyrosinase was 6.622×10⁻⁴ mM and the IC₅₀ for N-methyl nicotinic acid hydroxamate iodide for inhibiting tyrosinase was 0.5497 mM.

### Example 3

Tyrosinase Activity Assay In Situ

B16-F10 cells (1×10⁵) were placed on a 60 mm culture dish. After 12 hours, an adherent monolayer was formed on the bottom of the culture dish. After removal of the used culture medium, a fresh culture medium was added. A sample solution with various concentrations and/or 50 nM melanocytes stimulating hormone (MSH) were then added and cultivated at 37° C. for three days. After removal of the upper clean solution, the cells were washed two times using a PBS. After the cells were scraped away, protein was extracted and quantitated. One hundred μl of a phosphate buffer (250 mM, pH 6.5), 50 μl of a L-DOPA (10 mM) and 50 μl of a cell extract were sequentially added to a 96-well plate, uniformly mixed and reacted in a 37° C. culture box. The absorption of the result was then measured by an ELISA reader (475 nm).

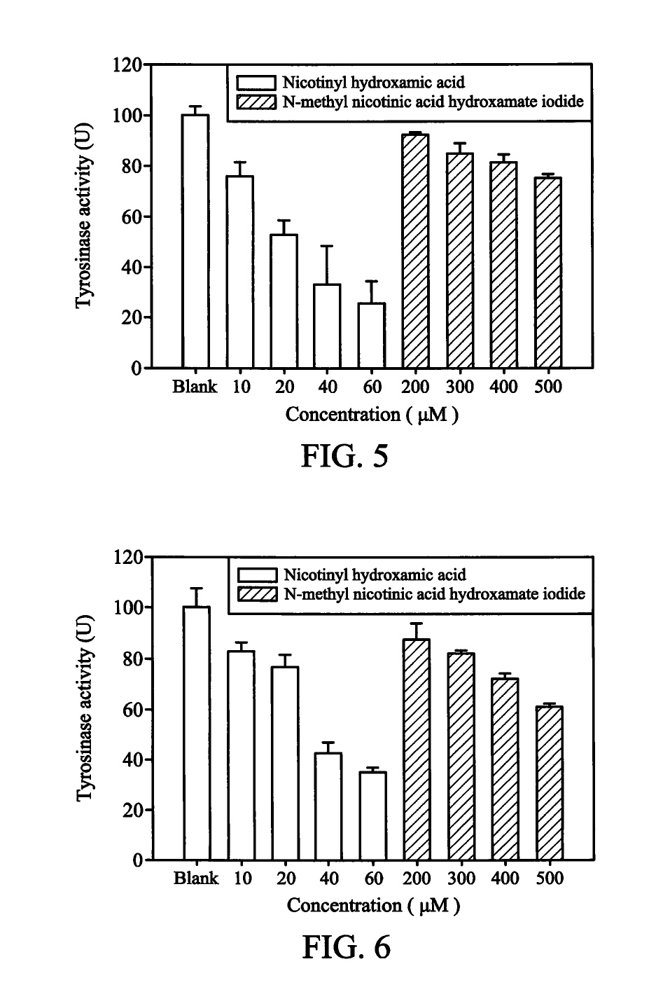

FIG. 5 shows a relationship between the nicotinyl hydroxamic acid concentration, the N-methyl nicotinic acid hydroxamate iodide concentration and tyrosinase activity (without addition of MSH). FIG. 6 shows a relationship between the nicotinyl hydroxamic acid concentration, the N-methyl nicotinic acid hydroxamate iodide concentration and tyrosinase activity (addition of MSH). The results indicated that when no MSH was added, as the nicotinyl hydroxamic acid concentration or the N-methyl nicotinic acid hydroxamate iodide concentration was increased, the tyrosinase activity of B16-F10 cells decreased. That is, inhibition to melanin was correlated with inhibition to tyrosinase by the nicotinyl hydroxamic acid and the N-methyl nicotinic acid hydroxamate iodide. When MSH was added, as the nicotinyl hydroxamic acid concentration or the N-methyl nicotinic acid hydroxamate iodide concentration was increased, the tyrosinase activity of B16-F10 cells was decreased. That is, inhibition to melanin was greatly correlated with inhibition to tyrosinase by the nicotinyl hydroxamic acid and the N-methyl nicotinic acid hydroxamate iodide.

### Example 4

Melanin Content Assay In Situ

B16-F10 cells (1×10⁵) were placed on a 60 mm culture dish. The nicotinyl hydroxamic acid and the N-methyl nicotinic acid hydroxamate iodide with various concentrations were respectively added and cultivated for three days in a culture box. After removal of the upper clean solution, the cells were washed two times using a PBS. The melanin was dissolved by 1N NaOH (90° C., 30 minutes). The melanin content was then measured (405 nm).

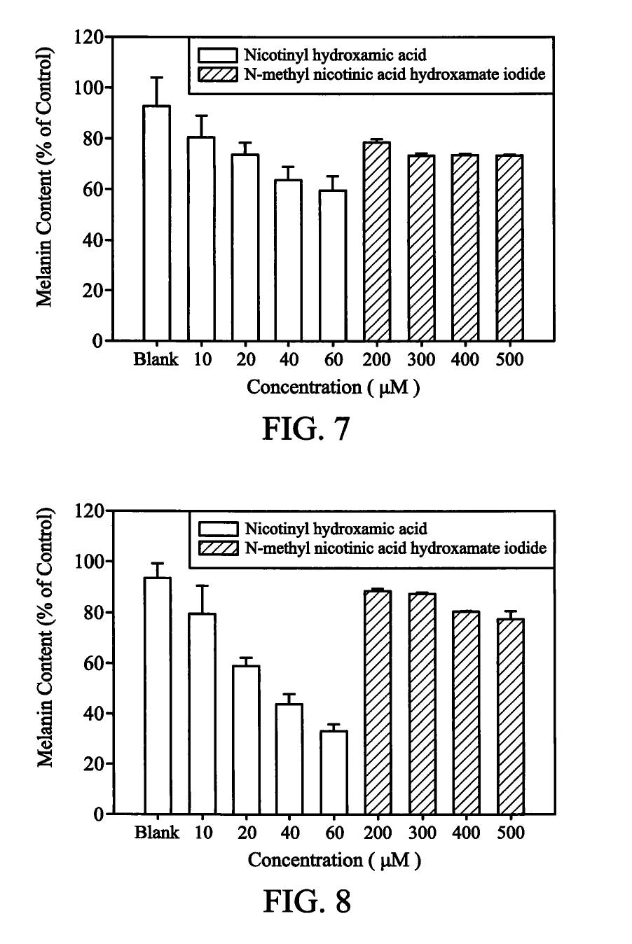

FIG. 7 shows a relationship between the nicotinyl hydroxamic acid concentration, the N-methyl nicotinic acid hydroxamate iodide concentration and melanin content (without addition of MSH). FIG. 8 shows a relationship between the nicotinyl hydroxamic acid concentration, the N-methyl nicotinic acid hydroxamate iodide concentration and melanin content (addition of MSH). The results indicated that the nicotinyl hydroxamic acid with various concentrations (10, 20, 40, 60 μM) effectively inhibited melanin of B16-F10 cells when the MSH was added and not added. The inhibition of melanin of B16-F10 cells was especially apparent when MSH was added. The N-methyl nicotinic acid hydroxamate iodide achieved the same effects.

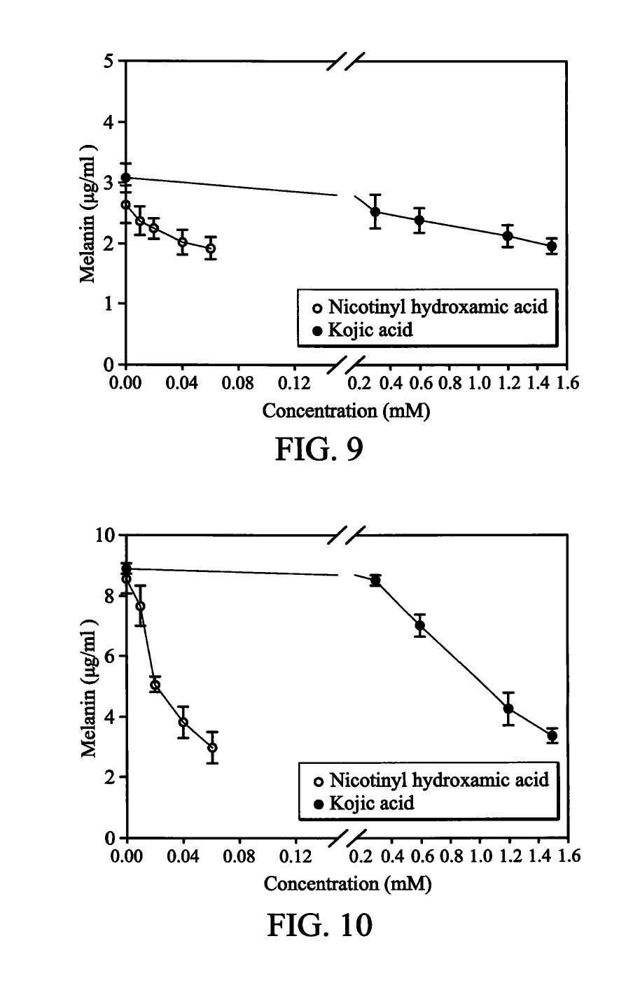

FIG. 9 shows a relationship between the nicotinyl hydroxamic acid concentration and melanin content (without addition of MSH) compared to kojic acid. FIG. 10 shows a relationship between the nicotinyl hydroxamic acid concentration and melanin content (addition of MSH) compared to kojic acid. The results indicated that when no MSH was added, to achieve the same inhibition effect, 0.06 mM of the nicotinyl hydroxamic acid was added, meanwhile, 1.5 mM of Kojic acid was added. That is, the added concentration of Kojic acid was 25 times that of the nicotinyl hydroxamic acid. When MSH was added, to achieve the same inhibition effect, 0.04 mM of the nicotinyl hydroxamic acid was added, meanwhile, 1.5 mM of Kojic acid was added. That is, the added concentration of Kojic acid was 37.5 times that of the nicotinyl hydroxamic acid.

### Example 5

Polyphenol Oxidase Inhibition Assay

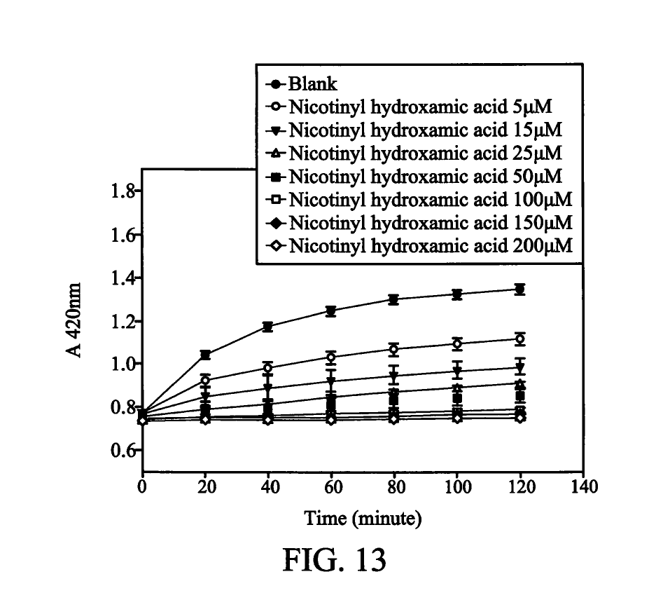

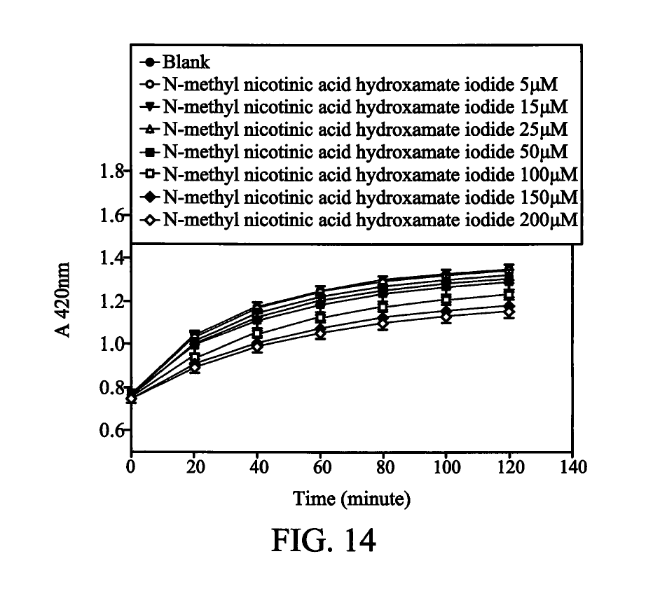

Apples were purchased from local supermarkets. 100 g of apples were peeled, cored and sliced prior to juicing in a blender. The preparation was with 100 ml distilled water filtered through swab and collected by centrifugation 12,000 rpm for 10 min at 4° C. The apple juice obtained was immediately collected 200 μl in a 96 well microplate and mixed nicotinyl hydroxamic acid and N-methyl nicotinic acid hydroxamate iodide (5-200 μM). At 20 intervals during 120 min, mixtures were examined spectrophotometrically at 420 nm, as shown in FIG. 13 (nicotinyl hydroxamic acid) and FIG. 14 (N-methyl nicotinic acid hydroxamate iodide).

The variation of the absorbance of the apple juice within two hours was measured by a spectrophotometer. The results indicated that as the time was increased, the absorbance at 420 nm of the blank was increased. However, as the concentrations of the nicotinyl hydroxamic acid and the N-methyl nicotinic acid hydroxamate iodide were increased (5 to 200 μM), the polyphenol oxidase in the apple juice was effectively inhibited. The inhibition to the polyphenol oxidase by the nicotinyl hydroxamic acid was greater than that by the N-methyl nicotinic acid hydroxamate iodide. Thus, both of the nicotinyl hydroxamic acid and the N-methyl nicotinic acid hydroxamate iodide can inhibit polyphenol oxidase, protecting polyphenol compounds with nutritive value of vegetables and fruits from catalysis.

### Example 6

Cell Viability Assay

The culture medium was removed from the culture dish filled with cells. The cells were washed using 10 ml of a PBS. The number of the cells was counted by a blood cell counter and diluted to a proper cell concentration. 500 l/well of the result was added to a 24-well culture plate and cultivated in a 37° C. culture box with 5% CO₂ and mist until the cells were stuck on the bottom of the culture dish. After removal of the used culture medium, a fresh drug-containing culture medium with various concentrations was added (the drug was directly added when cells were suspended) and cultivated for 24 hours in a 37° C. culture box with 5% CO₂ and mist. After removal of the upper clean solution, the cells were washed two times using a PBS. An MTT solution (500 μg/ml) prepared from the medium was then added to the well and reacted for 4-6 hours in a 37° C. culture box. After removal of the upper clean solution, 100 μl of a DMSO was added until a formazan crystal was dissolved. The absorption of the result was then measured by an ELISA reader (600 nm).

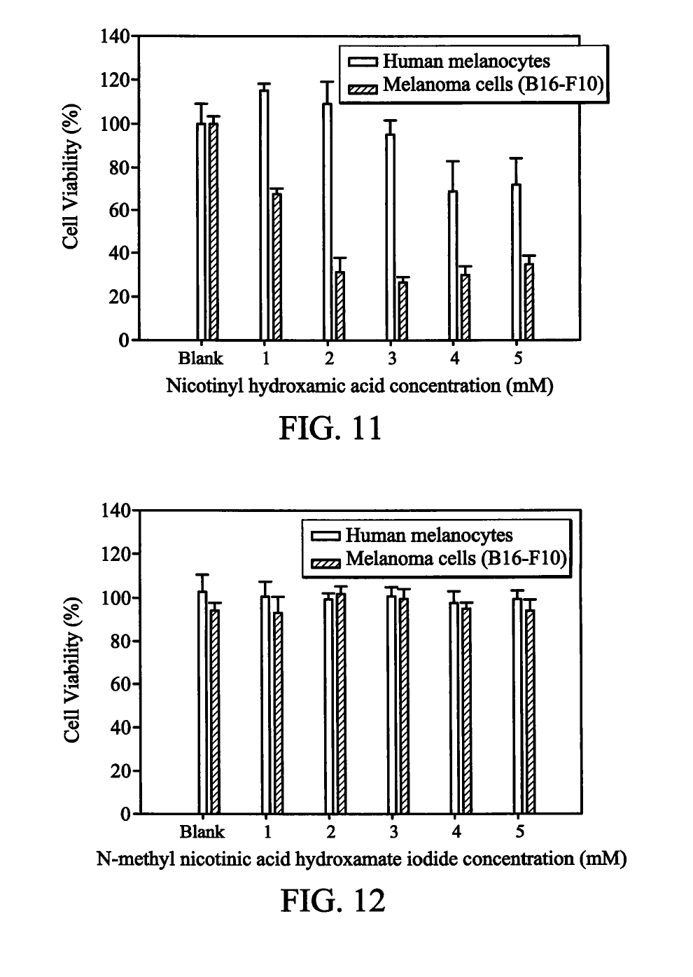

FIG. 11 shows a relationship between the nicotinyl hydroxamic acid concentration and cell viability of human melanocytes and melanoma cells (B 16-F10). The nicotinyl hydroxamic acid with various concentrations was added to the human melanocytes and melanoma cells (B16-F10) to assay the cell toxicity by a MTT method. The results indicated that the nicotinyl hydroxamic acid had low cell toxicity to the melanoma cells (B16-F10) under an effective concentration thereof to inhibit melanin of the B16-F10 cells. Further, when the concentration of the nicotinyl hydroxamic acid was increased to 1 mM, the human melanocytes were still alive.

FIG. 12 shows a relationship between the N-methyl nicotinic acid hydroxamate iodide concentration and cell viability of human melanocytes and melanoma cells (B16-F10). The N-methyl nicotinic acid hydroxamate iodide with various concentrations was added to the human melanocytes and melanoma cells (B16-F10) to assay the cell toxicity by a MTT method. The results indicated that the N-methyl nicotinic acid hydroxamate iodide had low cell toxicity to the melanoma cells (B16-F10) under an effective concentration thereof to inhibit melanin of the B16-F10 cells. Further, when the concentration of the N-methyl nicotinic acid hydroxamate iodide was increased to 5 mM, the human melanocytes and B16-F10 melanoma cells were still alive.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for scavenging free radicals comprising administering to a subject of effective amounts in a pharmaceutical composition comprising a compound of Formula (I) or (II) and a pharmaceutical acceptable salt, metal salt or solvent, 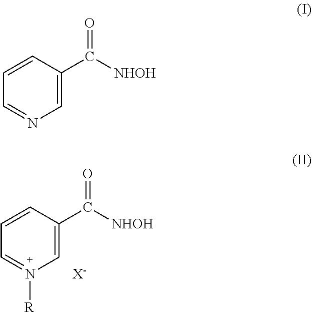
wherein R is C1-6 alkyl, vitamin C or sugar, and X is chlorine, bromine or iodine.

2. The method for scavenging free radicals as claimed in claim 1, wherein the subject is a human.

3. The method for scavenging free radicals as claimed in claim 1, wherein the pharmaceutical composition is for external use.

4. The method for scavenging free radicals as claimed in claim 1, wherein the pharmaceutical composition is for internal use.

5. A method for inhibiting tyrosinase comprising administering to a subject of effective amounts in a pharmaceutical composition comprising a compound of Formula (I) or (II) and a pharmaceutical acceptable salt, metal salt or solvent, 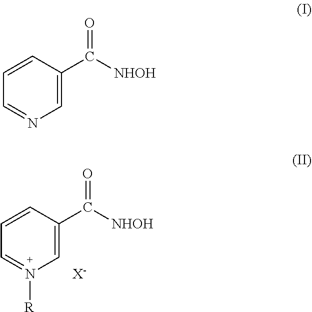
wherein R is C1-6 alkyl, vitamin C or sugar, and X is chlorine, bromine or iodine.

6. The method for inhibiting tyrosinase as claimed in claim 5, wherein the subject is a human.

7. The method for inhibiting tyrosinase as claimed in claim 5, wherein the pharmaceutical composition is for external use.

8. The method for inhibiting tyrosinase as claimed in claim 5, wherein the pharmaceutical composition is for internal use.

9. A method for inhibiting melanin comprising administering to a subject of effective amounts in a pharmaceutical composition comprising a compound of Formula (I) or (II) and a pharmaceutical acceptable salt, metal salt or solvent, 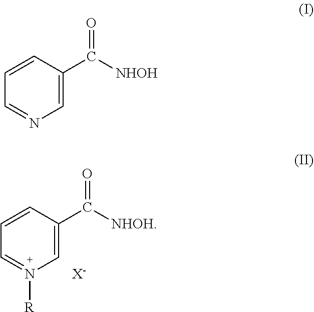
wherein R is C1-6 alkyl, vitamin C or sugar, and X is chlorine, bromine or iodine.

10. The method for inhibiting melanin as claimed in claim 9, wherein the subject is a human.

11. The method for inhibiting melanin as claimed in claim 9, wherein the pharmaceutical composition is for external use.

12. The method for inhibiting melanin as claimed in claim 9, wherein the pharmaceutical composition is for internal use.
